(19) United States
(12) Patent Application Publication
(10) Pub. No.: US 2015/0272469 A1
(43) Pub. Date: Oct. 1, 2015

(54) System and Methods For Combined Functional Brain Mapping

(71) Applicants: Michael Fox, Newton, MA (US); Hesheng Liu, Marblehead, MA (US)

(72) Inventors: Michael Fox, Newton, MA (US); Hesheng Liu, Marblehead, MA (US)

(21) Appl. No.: 14/672,690

(22) Filed: Mar. 30, 2015

Related U.S. Application Data
(60) Provisional application No. 61/973,185, filed on Mar. 31, 2014.

(51) Int. Cl. A61B 5/055; A61B 5/00

(52) U.S. Cl. CPC A61B 5/055; A61B 5/4064; A61B 5/742

(57) ABSTRACT

A system and methods for functional brain mapping is provided. The method includes providing a set of time-series functional magnetic resonance imaging (fMRI) data acquired from a brain of a subject while performing a functional task and decomposing the set of time-series fMRI data into a set of task signals and a set of non-task signals using a model related to the functional task performed by the subject. The method also includes generating a task activity map using the set of task signals and generating a non-task activity map using the set of non-task signals. The method further includes producing a combination map by selectively weighting the task activity map and non-task activity map, and combining the selectively weighted maps, using a statistical parameter, in dependence of a threshold.

### CROSS REFERENCE TO RELATED APPLICATIONS

This application claims the benefit of U.S. Provisional Patent Application Ser. No. 61/973,185, filed on Mar. 31, 2014, and entitled “SYSTEM AND METHODS FOR COMBINED FUNCTIONAL BRAIN MAPPING.”

### STATEMENT REGARDING FEDERALLY SPONSORED RESEARCH

This invention was made with government support under NS069805 awarded by the National Institutes of Health. The government has certain rights in the invention.

### BACKGROUND

The present disclosure relates generally to systems and methods for functional magnetic resonance imaging and, in particular, to systems and methods directed to brain mapping using task-based and spontaneous functional activity.

Functional magnetic resonance imaging (fMRI) has demonstrated great clinical utility in areas associated with neurosurgical interventional procedures, facilitating brain mapping for guidance in surgery. Traditionally, pre-operative mapping employs intermittent task periods to identify brain areas to be avoided during surgery. For example, the primary motor cortex may be activated by instructing a subject to perform finger tapping. First used for pre-operative mapping almost 20 years ago, this task-based strategy continues to dominate clinical practice. FMRI maps obtained using this approach correlate with intra-operative electrophysiology, Wada testing, and loss of function post-operatively. However, pre-operative mapping patients frequently lack the ability to perform tasks well due to age or disability, and maps are frequently confounded by artifacts, accuracy and clinical utility, with wide variation across different patients and investigative studies. Moreover, task-based mapping utilizes only a small percentage of total fMRI variance.

A mapping approach that circumvents some of these limitations using spontaneous brain activity that occurs while a subject is at rest. Termed resting state functional connectivity (rs-fcMRI), this technique employs fluctuations in brain activity across separate brain regions, identifying patterns of intercorrelation between the functioning of these regions and affording quantitative indices of resting-state functional connectivity. Since spontaneous activity mapping does not necessitate subject participation, it may be performed when subjects are asleep or sedated. Therefore, due to its simplicity and the reliability of rs-fMRI data, this imaging modality presents increased potential for clinical application, with important ramification in pre-operative planning for patients with neurosurgical conditions, such as epileptic or cancer patients.

Many investigations have reported correlation between rs-fcMRI and traditional task-based functional mapping results, as well as intra-operative cortical stimulation approaches. There has been no evidence to conclusively demonstrate whether or the extent to which functional information generated by these approaches is related, independent or redundant.

Compared to traditional fMRI approaches rs-fcMRI may be advantageous, due to less burden on account of experimental design, subject compliance and training demands. However, rs-fcMRI data is confounded by different but equally problematic artifacts on account of physiological and motion sources. In addition, there has been a continued reticence to abandon task-based mapping in favor of rs-fcMRI, for purposes of pre-operative planning, since many clinical patients are able perform tasks at least partially. Although generally it may be possible to evaluate information from both spontaneous activity and task-based mapping, acquiring both imaging types may not be practical on account of an increased scanning time. This would be undesirable from the perspective of cost, and patient convenience, as well as data quality since movement artifacts increase as a function of the duration that patients are in the scanner.

There is a need for systems and methods to improve brain mapping of cognitive, language, or motor function based on both task-related as well as spontaneous (non-task-related) brain activity. Further, there is a need to perform this mapping in a way that does not extend the time or cost of the data acquisition itself.

### SUMMARY OF THE INVENTION

The present invention overcomes the aforementioned drawbacks by providing a system and methods directed to functional mapping a brain of a subject using task-related and non-task-related brain activities. Specifically, a novel approach is introduced using the concept that fMRI data signals acquired during a functional task include a superposition of underlying coherent non-task signals, resulting from fluctuations, or spontaneous brain activity, and the task-induced signals. Using acquired time-series fMRI data, task and non-task activities may be then separated to generate different functional maps based on their respective task and non-task signals, which may subsequently be combined using a selective weighting to achieve more robust mapping results.

In accordance with one aspect of the disclosure, a method for functional brain mapping is provided. The method includes providing a set of time-series functional magnetic resonance imaging (fMRI) data acquired from a brain of a subject while performing a functional task and decomposing the set of time-series fMRI data into a set of task signals and a set of non-task signals using a model related to the functional task performed by the subject. The method also includes generating a task activity map using the set of task signals and generating a non-task activity map using the set of non-task signals. The method further includes producing a combination map by selectively weighting the task activity map and non-task activity map, and combining the selectively weighted maps, using a statistical parameter, in dependence of a threshold.

In accordance with an other aspect of the disclosure, a system for functional brain mapping is provided. The system includes an input configured to receive a set of time-series functional magnetic resonance imaging (fMRI) data acquired from a brain of a subject while performing an activity, and at least one processor configured to decompose the set of time-series fMRI data into a set of task signals and a set of non-task signals using a model related to the functional task performed by the subject. The at least one processor is also configured to generate a task activity map using the set of task signals, generate a non-task map using the set of non-task signals, and produce a combination map by selectively weighting the task activity map and non-task activity map, and combining the selectively weighted maps using a statistical parameter, in dependence of a threshold. The at least one processor is further configured to generate, using the combined map, a report indicative a region of interest in the brain of the subject. The system also includes an output configured to display the report.

In accordance with one aspect of the disclosure, a method for functional brain mapping is provided. The method includes providing a set of time-series functional magnetic resonance imaging (fMRI) data acquired from a brain of a subject while performing a functional task and separating from the set of time-series fMRI data a set of task signals using a model related to the functional task performed by the subject. The method also includes generating a task activity map using the set of task signals, and providing a non-task map. The method further includes producing a combination map by selectively weighting the task activity map and non-task map, and combining the selectively weighted maps, using a statistical parameter, in dependence of a threshold.

The foregoing and other advantages of the invention will appear from the following description.

### BRIEF DESCRIPTION OF THE DRAWINGS

FIG. 1 is a schematic of an example system for use in performing combination mapping of brain function of a subject, in accordance with the present invention.

FIG. 2 is a flowchart setting forth steps for a method of performing combination mapping using functional magnetic resonance imaging (“fMRI”) data.

FIG. 3 is a block diagram of an example of a MRI system.

FIG. 4 is a schematic depicting a combination mapping method, in accordance with the present invention, performed by decomposing and combining task and non-task signal data.

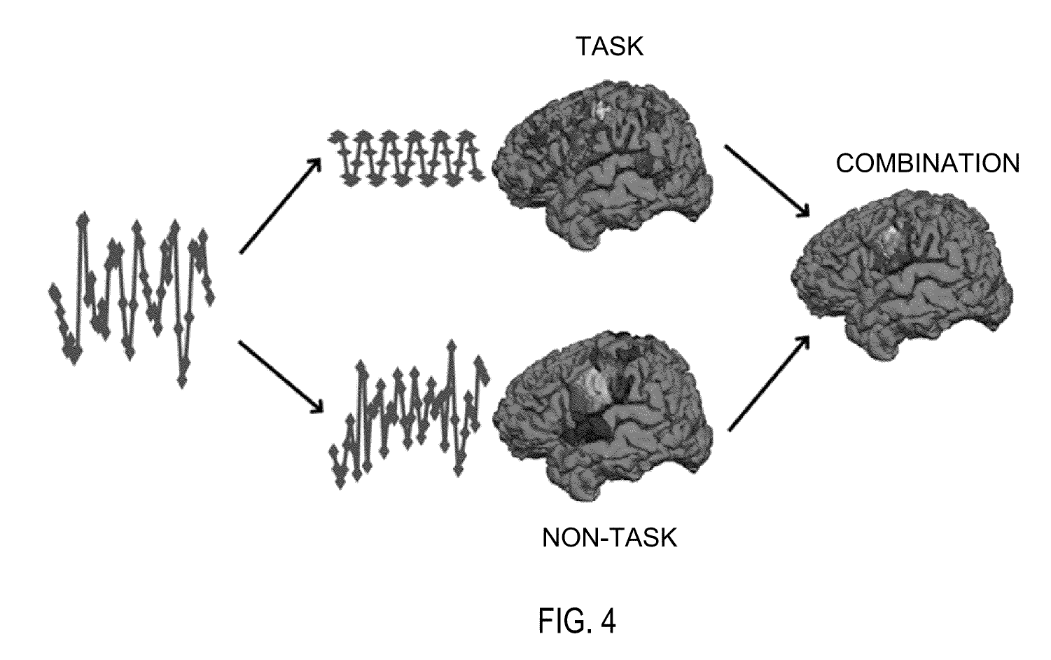

FIG. 5 is a graphical example comparing functional brain mapping using electrical cortical stimulation, traditional task activity mapping and a combination mapping method, in accordance with the present invention.

FIGS. 6A-B are a graphical example illustrating that a combination mapping method, in accordance with the present invention, provides improved sensitivity, specificity and signal to noise as compared to traditional task activity mapping.

FIG. 7 is a graphical example illustrating mapping for a plurality of functional networks.

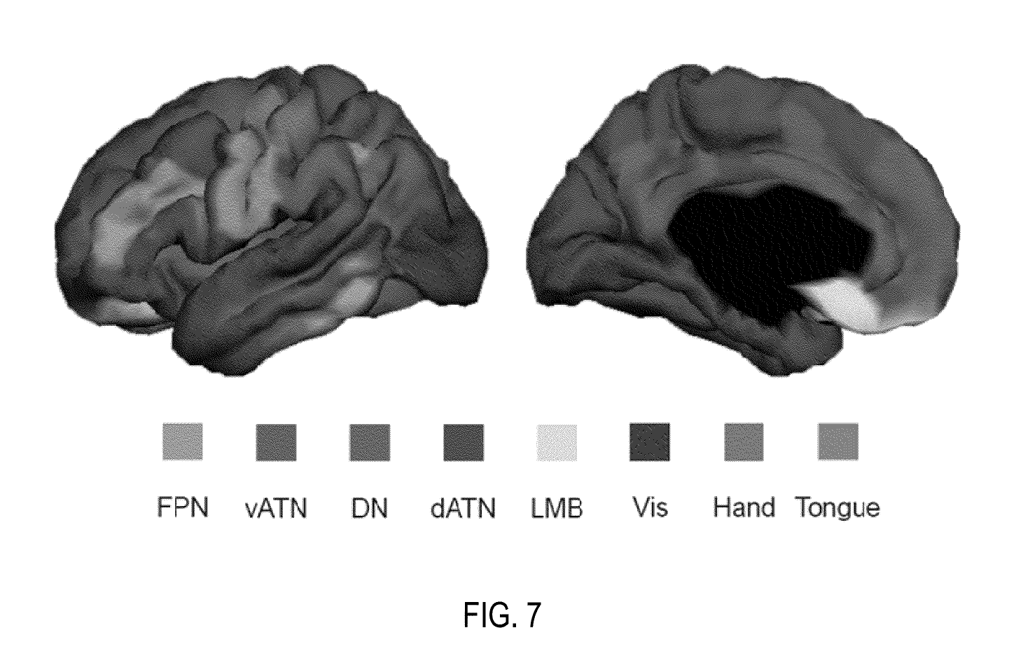

FIG. 8 is a graphical example comparing functional brain mapping for several subjects using electrical cortical stimulation, traditional task activity mapping and a combination mapping method.

FIGS. 9A-B are a graphical example illustrating a comparison of sensitivity and specificity profiles for several mapping approaches.

### DETAILED DESCRIPTION

Pre-operative brain mapping with fMRI is often used to guide neurosurgical procedures with the goal of avoiding damage to critical areas such as primary motor cortex. The principle approach for generating these maps relies on brain responses evoked by one or more functional tasks and, despite known limitations, has dominated clinical practice for almost 20 years. Recently, pre-operative mapping based on correlations in spontaneous brain activity has been demonstrated, however this approach has its own limitations and has not seen widespread clinical use.

Described herein are systems and methods for generating a combined map of task-related and non-task-related functional activity using a magnetic resonance imaging (“MRI”) system. It is an aspect of the present invention that spontaneous, or non-task-related, activity mapping provides complimentary information to task-related functional activation mapping. The same pre-operative fMRI dataset can be used to derive both task-related and spontaneous activity maps, which can subsequently be combined to significantly improve signal-to-noise ratio. The combination mapping algorithm described herein provides a significant benefit in both sensitivity and specificity without requiring additional scan time or modification to conventional pre-operative mapping data acquisition protocols.

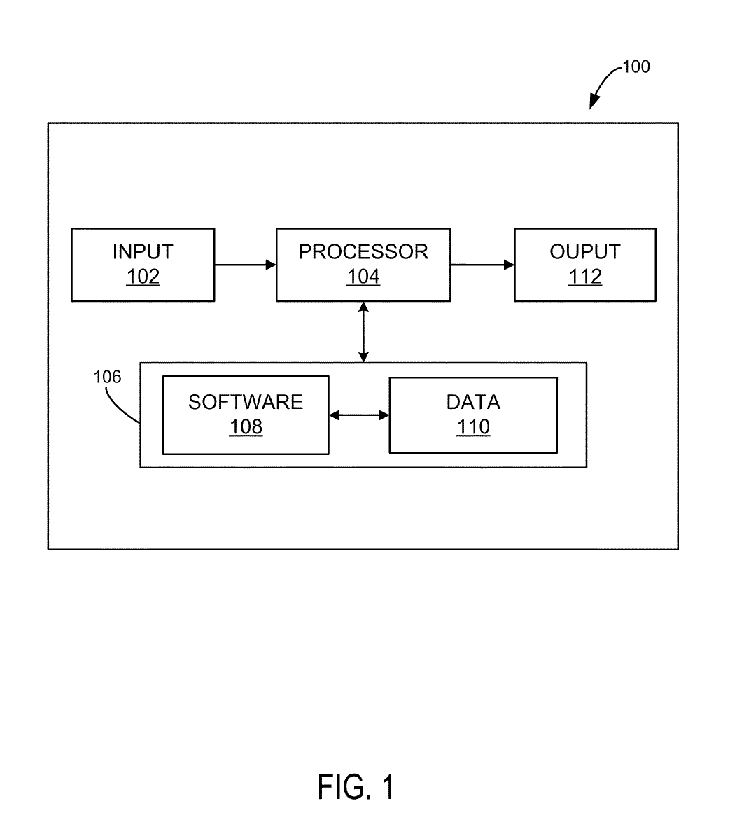

Turning to FIG. 1, a block diagram is shown of an example of a system 100 for generating functional mapping of a subject's brain. The system 100 generally may include an input 102, at least one processor 104, a memory 106, an output 108, and any device for reading computer-readable media (not shown). The system 100 may be, for example, a workstation, a notebook computer, a personal digital assistant (PDA), a multimedia device, a network server, a mainframe or any other general-purpose or application-specific computing device. The system 100 may operate autonomously or semi-autonomously, or may read executable software instructions from a computer-readable medium (such as a hard drive, a CD-ROM, flash memory and the like), or may receive instructions from a user, or any another source logically connected to computer or device, such as another networked computer or server, via the input 102. As will become apparent, system 100 may find application in a variety of clinical and research settings, including pre-operative functional brain mapping protocols and procedures.

The input 102 may take any suitable shape or form, as desired, for operation of the system 100, including the ability for selecting, entering or otherwise specifying parameters consistent with performing tasks, processing or operating the system 100. In some aspects, the input 102 may be configured to receive data, such as functional magnetic resonance image (fMRI) data, or maps, associated with resting states, task states, and any other states of consciousness of one or more subjects. Such data may be pre-processed, filtered and corrected using known methods and technologies. For example, data may be processed to include correction for section-dependent time shifts, intensity differences, motion, or any other nuissance regressors. In addition, the input 102 may also be configured to receive any other data or information considered useful for functional localization. For example, the input 102 may also receive anatomical data, or maps, for use by the system 100.

Among the processing tasks for operating the system 100, the at least one processor 104 may also be configured to receive raw or processed data, such as fMRI data. In some configurations, the at least one processor 104 may also be configured to carry out any number of post-processing steps on data received by way of the input 102, including manipulating, filtering, enhancing, or correcting the data in relation to artifacts or errors, due to, for example, scanner characteristics, subject motion, physiological sources, and so forth. In addition, the at least one processor 104 may be capable of performing computations using signals derived from raw or processed data. For example, the at least one processor 104 may be capable of decomposing time-series fMRI data, obtained by way of the input 102, into a set of task and non-task signals using a model, such as a general linear model, in dependence of the functional task performed by the subject(s).

The at least one processor 104 may also be capable of generating task and non-task activity maps using signals separated or decomposed from received or processed data, such as time-series fMRI data. Additionally, the at least one processor 104 may also be configured to produce a combination of maps by selectively weighting received or generated task and non-task maps, which may be functional and/or anatomical maps. As will be described, such combinations may be carried out by the at least one processor 104, using a selective weighting approach that employs statistical parameters such as t-values, and other statistical parameters determined using mapping and data obtained from the subject(s). In some aspects, the at least one processor 104 may be further configured to perform a parametric optimization process to determine optimal weighting of each map in the combined map.

The memory 106 may contain software 110 and data 112, and may be configured for storage and retrieval of processed information, instructions and data to be processed by the processor 104. In some aspects, the software 110 may contain instructions directed to producing combined mapping, as mentioned, and instructions for determining optimal weighting for maps included in the combined map. Also, the data 112 may take include any data necessary for operating the system 100, and may include any raw or processed information in relation to subject data and map(s).

In addition, the output 108 may take any shape or form, as desired, and may be configured for displaying, in addition to other desired information, any decomposed, separated or combined maps. For example, the output 108 may be configured to display indices, metrics, parameters, maps, or any combinations thereof, including any information indicative of regions of interest in subject brain(s), such as functional brain networks or functional organization.

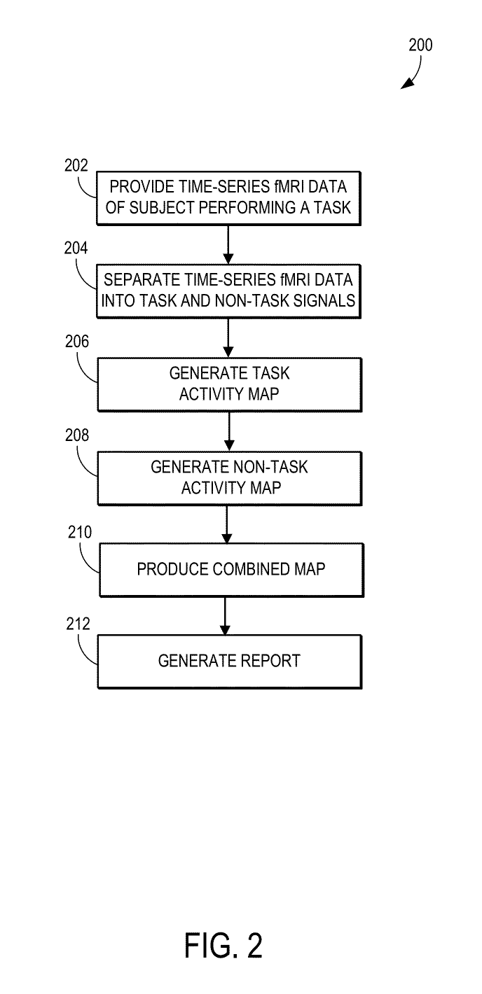

Turning to FIG. 2, a flowchart is shown, illustrating an example of a process 200 for providing functional brain mapping using fMRI data acquired from a subject while performing a functional task, in accordance with some embodiments of the present invention. The process begins at process block 202 where time-series fMRI data is provided. As mentioned, the time-series fMRI data may be pre-processed, post-processed or manipulated in any manner desirable. In some aspects, the fMRI data may also be acquired at process block 202 using a magnetic resonance system (“MRI”), as will be described. Preferably, the time-series fMRI data is task-related data acquired while the subject performs a functional task.

Then, at process block 204, the provided or acquired time-series fMRI data is decomposed, or separated, into task and non-task signals, using any suitable models, such as simple regression models or more sophisticated general linear models. As such, the task and non-task signals may then be used to generate task or non-task activity maps at process blocks 206 and 208 using methods and technologies known in the art, such as seed-based analyses, independent-component analyses, clustering algorithms, multivariate pattern classifications, and so forth. In some alternative configurations, at process block 208, non-task activity maps, or other maps, may be provided via the input 102. As an example, a non-task activity map generated from resting-state fMRI data acquired from the subject may be provided. In some instances, separately acquired resting-state fMRI data may more accurately represent a non-task condition than the decomposed non-task signals.

At process block 210 a combined map is produced. In particular, task and non-task activity maps may be combined by way of selective weighting determined using subject data. Such weighing may be determined using a measure of robustness for the generated task activity maps, in dependence of pre-selected or computed thresholds, where the robustness measure may provide an indication of how well a subject performs a task, or a significance of task-induced activation. As one example, a measure of robustness may be obtained via a statistical parameter computed using generated task activity maps. As one example, the statistical parameter may include a median t-value or an average t-value for a number of desired locations, or vertices, within a region-of-interest in the brain, which can correspond to a functional network of the subject's brain. As another example, the statistical parameter can be an average t-value of all voxels greater than two standard deviations from the mean.

Therefore, in some aspects of the present invention, when the statistical parameter is larger than a pre-selected or computed threshold, the task activity map may be considered robust, and thus the combination map may be generated by having task and non-task activity maps weighted equally. By contrast, when the statistical parameter is smaller than the pre-selected or computed threshold, in some embodiments, the non-task activity map may be weighted more heavily than the task activity map according to the following equation:

$$CM = ( 1 - \frac{T}{2 \cdot th} ) \times SAM + ( \frac{T}{2 \cdot th} ) \times TAM ; \qquad ( 1 )$$

where CM is the combination map; SAM is the spontaneous, or non-task, activity map; TAM is the task-activity map; T is the statistical parameter, as described above; and th is the threshold value. As an example, the threshold value can be th=5. It is noteworthy that in the limiting case when subjects are unable to perform the task, the combination map computed in Eqn. 1 becomes equivalent to the non-task activity map.

As mentioned, the threshold may be pre-selected, or determined, for example, by computing a median t-value for a plurality of locations, or vertices, using the task activity map. Alternatively, the threshold may be determined by way of a parametric optimization process in order to establish an optimal weighting of maps included in the combination map of Eqn. 1, in dependence of subject data. Therefore, the selective weighting approach as described allows for variation across subjects, since likely task activity maps differ between subjects.

In other envisioned configurations, task activity maps, generated at process block 206 using separated task signals, may also be combined with other non-task maps using a selective weighting approach, as detailed above. For example, task activity maps could be combined with anatomical maps to improve mapping accuracy. In one approach, an anatomical parcellation, which may be generated using a surface registration technique, may be used as subject-specific masks. In another approach, seed regions may be obtained by a functional connectivity analysis and those seed regions may be employed to obtain anatomical masks.

Finally, at process block 210 a report is generated, which may take any shape or form as desired, including indices, metrics, parameters, maps, or any combinations thereof. For example, a generated report may be in the form of two or three-dimensional combination maps, indicative of a functional networks in relation to anatomical features, portions or perspectives of the brain of the subject.

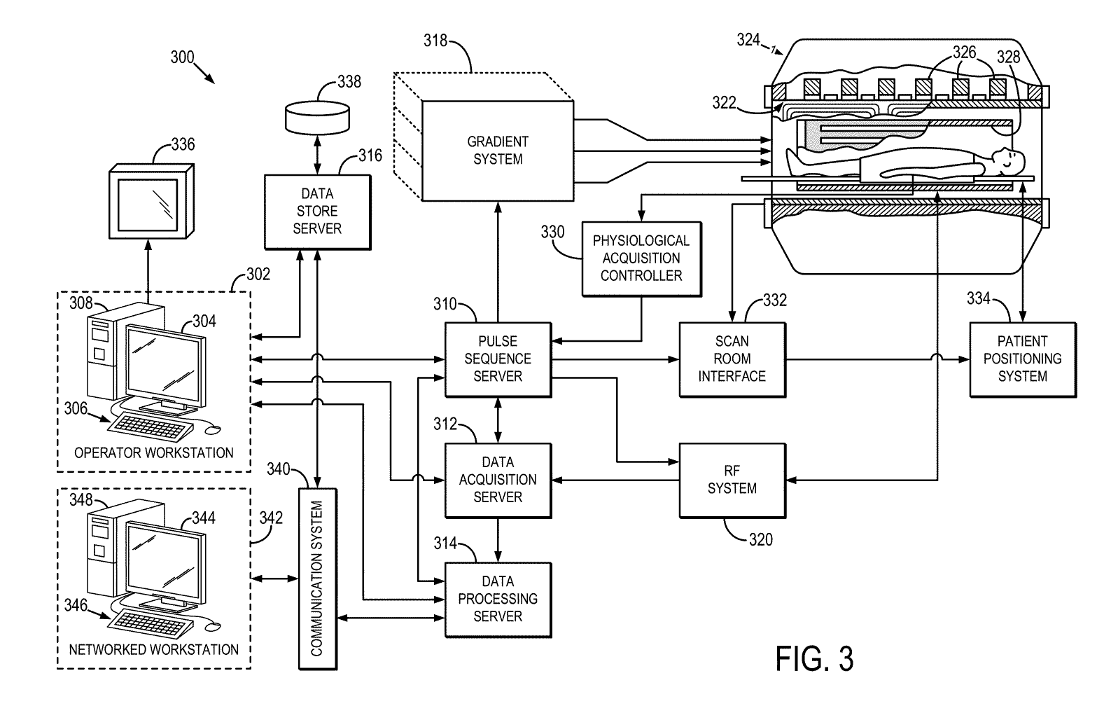

Referring particularly now to FIG. 3, an example of a MRI system 300 is illustrated, for use in accordance with the present invention. The MRI system 300 includes an operator workstation 302, which will typically include a display 304; one or more input devices 306, such as a keyboard and mouse; and a processor 308. The processor 308 may include a commercially available programmable machine running a commercially available operating system. The operator workstation 302 provides the operator interface that enables scan prescriptions to be entered into the MRI system 300. In general, the operator workstation 302 may be coupled to four servers: a pulse sequence server 310; a data acquisition server 312; a data processing server 314; and a data store server 316. The operator workstation 302 and each server 310, 312, 314, and 316 are connected to communicate with each other. For example, the servers 310, 312, 314, and 316 may be connected via a communication system 340, which may include any suitable network connection, whether wired, wireless, or a combination of both. As an example, the communication system 340 may include both proprietary or dedicated networks, as well as open networks, such as the internet.

The pulse sequence server 310 functions in response to instructions downloaded from the operator workstation 302 to operate a gradient system 318 and a radiofrequency (“RF”) system 320. Gradient waveforms necessary to perform the prescribed scan are produced and applied to the gradient system 318, which excites gradient coils in an assembly 322 to produce the magnetic field gradients Gₓ, G_{y}, and G_{z}, used for position encoding magnetic resonance signals. The gradient coil assembly 322 forms part of a magnet assembly 324 that includes a polarizing magnet 326 and a whole-body RF coil 328.

RF waveforms are applied by the RF system 320 to the RF coil 328, or a separate local coil (not shown in FIG. 3), in order to perform the prescribed magnetic resonance pulse sequence. Responsive magnetic resonance signals detected by the RF coil 328, or a separate local coil (not shown in FIG. 3), are received by the RF system 320, where they are amplified, demodulated, filtered, and digitized under direction of commands produced by the pulse sequence server 310. The RF system 320 includes an RF transmitter for producing a wide variety of RF pulses used in MRI pulse sequences. The RF transmitter is responsive to the scan prescription and direction from the pulse sequence server 310 to produce RF pulses of the desired frequency, phase, and pulse amplitude waveform. The generated RF pulses may be applied to the whole-body RF coil 328 or to one or more local coils or coil arrays (not shown in FIG. 3).

The RF system 320 also includes one or more RF receiver channels. Each RF receiver channel includes an RF preamplifier that amplifies the magnetic resonance signal received by the coil 328 to which it is connected, and a detector that detects and digitizes the I, and Q quadrature components of the received magnetic resonance signal. The magnitude of the received magnetic resonance signal may, therefore, be determined at any sampled point by the square root of the sum of the squares of the I and Q components:

M=\sqrt{I²+Q²} (2);

and the phase of the received magnetic resonance signal may also be determined according to the following relationship:

$$ϕ = {tan}^{- 1} ( \frac{Q}{I} ) . \qquad ( 3 )$$

The pulse sequence server 310 also optionally receives patient data from a physiological acquisition controller 330. By way of example, the physiological acquisition controller 330 may receive signals from a number of different sensors connected to the patient, such as electrocardiograph (“ECG”) signals from electrodes, or respiratory signals from a respiratory bellows or other respiratory monitoring device. Such signals are typically used by the pulse sequence server 310 to synchronize, or “gate,” the performance of the scan with the subject's heart beat or respiration.

The pulse sequence server 310 also connects to a scan room interface circuit 332 that receives signals from various sensors associated with the condition of the patient and the magnet system. It is also through the scan room interface circuit 332 that a patient positioning system 334 receives commands to move the patient to desired positions during the scan.

The digitized magnetic resonance signal samples produced by the RF system 320 are received by the data acquisition server 312. The data acquisition server 312 operates in response to instructions downloaded from the operator workstation 302 to receive the real-time magnetic resonance data and provide buffer storage, such that no data is lost by data overrun. In some scans, the data acquisition server 312 does little more than pass the acquired magnetic resonance data to the data processor server 314. However, in scans that require information derived from acquired magnetic resonance data to control the further performance of the scan, the data acquisition server 312 is programmed to produce such information and convey it to the pulse sequence server 310. For example, during prescans, magnetic resonance data is acquired and used to calibrate the pulse sequence performed by the pulse sequence server 310. As another example, navigator signals may be acquired and used to adjust the operating parameters of the RF system 320 or the gradient system 318, or to control the view order in which k-space is sampled. In still another example, the data acquisition server 312 may also be employed to process magnetic resonance signals used to detect the arrival of a contrast agent in a magnetic resonance angiography (“MRA”) scan. By way of example, the data acquisition server 312 acquires magnetic resonance data and processes it in real-time to produce information that is used to control the scan.

The data processing server 314 receives magnetic resonance data from the data acquisition server 312 and processes it in accordance with instructions downloaded from the operator workstation 302. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 314 are conveyed back to the operator workstation 302 where they are stored. Real-time images are stored in a data base memory cache (not shown in FIG. 3), from which they may be output to operator display 312 or a display 336 that is located near the magnet assembly 324 for use by attending physicians. Batch mode images or selected real time images are stored in a host database on disc storage 338. When such images have been reconstructed and transferred to storage, the data processing server 314 notifies the data store server 316 on the operator workstation 302. The operator workstation 302 may be used by an operator to archive the images, produce films, or send the images via a network to other facilities.

The MRI system 300 may also include one or more networked workstations 342. By way of example, a networked workstation 342 may include a display 344; one or more input devices 346, such as a keyboard and mouse; and a processor 348. The networked workstation 342 may be located within the same facility as the operator workstation 302, or in a different facility, such as a different healthcare institution or clinic.

The networked workstation 342, whether within the same facility or in a different facility as the operator workstation 302, may gain remote access to the data processing server 314 or data store server 316 via the communication system 340. Accordingly, multiple networked workstations 342 may have access to the data processing server 314 and the data store server 316. In this manner, magnetic resonance data, reconstructed images, or other data may exchanged between the data processing server 314 or the data store server 316 and the networked workstations 342, such that the data or images may be remotely processed by a networked workstation 342. This data may be exchanged in any suitable format, such as in accordance with the transmission control protocol (“TCP”), the internet protocol (“IP”), or other known or suitable protocols.

The above-described systems and methods may be further understood by way of example. The examples provided below are for illustrative purposes only, and are not intended to limit the scope of the invention in any way. Indeed, various modifications of the invention in addition to those shown and described herein will become apparent to those skilled in the art from the foregoing description and the following example falls within the scope of the appended claims. For example, certain arrangements and configurations are presented, although it may be understood that other configurations may be possible, and still considered to be within the scope of the present invention.

### Example I

### Methods

### Participants

Eight surgical candidates with intractable epilepsy participated in the study. The experiment included a pre-operative fMRI scan, surgical implantation of subdural electrode grids, and direct electrical cortical stimulation (ECS) using these grids. No seizures were observed one hour before or after the fMRI or ECS in all patients.

### MRI Data Acquisition

MRI data was collected on a 3.0 Tesla MRI scanner using an 8-channel head coil. Structural images were acquired using a sagittal magnetization-prepared rapid gradient echo T1-weighted sequence, and functional data were collected using an echo planar imaging sequence.

Two types of functional runs were collected, task activation runs (all eight subjects) and resting state or spontaneous activity runs (six of eight subjects). Task activation runs included one of three types of self-paced movements (left hand, right hand, or tongue) consistent with standard pre-operative mapping paradigms. Each task run was 144 second long and included six 12-second task blocks interleaved with six 12-second rest intervals. Subjects performed motor tasks according to the instructions presented on the computer screen using the Psychophysics Toolbox in MATLAB (The MathWorks, Inc.).

Six of the eight patients also had resting state fMRI data collected in addition to the above task data. Each of these subjects performed two resting-state runs (360 s each run), during which subjects were asked to fixate on a crosshair in the center of the screen. These pure resting state runs were collected for comparison purposes with the maps created based on spontaneous activity extracted from the task runs.

### Electrical Cortical Stimulation Data Acquisition

After an adequate number of seizures had been recorded, direct electrical cortical stimulation mapping was performed at the bedside to identify motor and somatosensory cortices. Using an Ojemann Cortical Stimulator (Integra Life-Sciences), trains of 60-Hz biphasic pulses lasting for 2-5 seconds were delivered to selected pairs of electrodes. The current intensity of the stimulation started at 2 mA and was gradually increased until patients showed or reported symptoms related to the sensory motor cortex or the stimulus strength reached 15 mA. As each stimulation involved a pair of electrodes, both electrodes were considered positive when a hand or tongue movement or sensation was produced.

### Registering Intracranial Electrodes to Cortical Surface and ROI Definition

A post-implantation CT scan was obtained within 24-48 h after the implant surgery for localizing the electrodes. The post-implantation CT images were registered to the T1-weighted MRI images with a mutual-information-based linear transform algorithm. Cortical surfaces were reconstructed from high-resolution T1-weighted images using the Freesurfer 5.0 pipeline. To facilitate the extraction of electrode coordinates, the 3D pial surface was overlaid with semitransparent CT images using an in-house visualization toolbox. The effects of surgical intervention may cause the exposed brain to move away from the skull and some of the electrodes extracted from post-implantation CT images may appear off the surface reconstructed from pre-surgical MR images. In order to correct this non-linear distortion of the brain surface, these electrodes' locations were manually adjusted according to the grid shape and other electrodes on the 3D pial surface. This manual adjustment was done prior to the functional MRI data processing and with no knowledge of subsequent functional information. MRI surface vertices within a 6 mm radius of electrodes associated with hand or tongue movements were defined as positive while vertices within 6 mm of other electrodes were defined as negative. This mask was smoothed with a 6 mm full-width-half-maximum kernel, resulting in an ECS map on the surface that could be directly compared to maps obtained using fMRI data. This ECS surface map was also used to generate hand or tongue regions of interest for the analysis of fMRI signal to noise ratios.

### MRI Data Analysis

MRI data were processed in surface-space using previously described procedures. Structural data was processed using the FreeSurfer version 4.5.0 software package (http://surfer.nmr.mgh.harvard.edu). Surface mesh representations of the cortex from each individual subject's structural images were reconstructed and registered to a common spherical coordinate system. The structural and functional images were aligned using boundary-based registration. The BOLD fMRI data were then aligned to the common spherical coordinate system via sampling from the middle of the cortical ribbon in a single interpolation step.

Conventional task-evoked activation maps were estimated using the general linear model as implemented in SPM2 (Wellcome Department of Cognitive Neurology, London, UK). Regressors of no interest included motion correction parameters and low frequency drift. Task induced BOLD response was modeled by convolving the hemodynamic response function with the experimental design. The significance of the task activation at each vertex was calculated in SPM2 using a t-test.

Maps based on spontaneous activity were constructed from two sources, namely pure resting state runs, which consisted entirely of spontaneous activity, and residual spontaneous activity underlying task-evoked activity from the task runs. In both cases, data were band-passed filtered (0.01-0.08 Hz) and several sources of spurious or regionally nonspecific variance were regressed out. Nuisance regressors included six-parameter rigid body head motion obtained from motion correction, the signal averaged over the whole brain (the global signal), the signal averaged over the lateral ventricles, and the signal averaged over a region centered in the deep cerebral white matter. For processing of the task-based data, an extra nuisance regressor was included consisting of the modeled task-related response. The data from all three types of movement tasks were concatenated for spontaneous activity analysis.

Functional maps based on spontaneous activity were generated using a parcellation approach that segments the cortex into distinct functional areas based on the correlation of each brain vertex with multiple pre-set regions of interest. As an example, the pre-set regions of interest can include 17 regions per hemisphere, or 34 regions total, divided into 8 functional networks. These regions and networks can be determined from previously acquired cortical parcellation data.

In one example, for each brain vertex in the diseased hemisphere, correlations to the 17 regions in the opposite healthy hemisphere were calculated. Only cross-hemisphere correlations to the healthy hemisphere were used to render the technique robust to local perturbations in anatomy. These 17 regional correlation values were reduced to 8 network correlation values by averaging the results from multiple regions within a network, as described above. Each vertex was identified as part of the network to which it showed the strongest positive correlation. The intensity of each vertex within a network is the ratio of the vertex's correlation with that network over the vertex's correlation to the next highest network. As such, voxels with the highest values are those that are significantly correlated to one network and not to other networks. This cortical parcellation approach can thus used in favor of the simpler seed-based mapping as preliminary analyses suggested that parcellation was less susceptible to artifact and provided more distinct and reproducible cortical boundaries in individual subject.

The task-evoked activation map and spontaneous activity map produced above were combined into a single functional map using a selective weighting method, as described above. The robustness of the task-activation map for each subject was quantified by averaging the t-values of the most activated vertices, which are defined as the vertices where the t-value is more than two standard deviations above the mean. If the mean t-value of these top vertices was larger than a specific threshold, the map was considered robust and the task-evoked and spontaneous activity maps were weighted equally. If the average t-value of the top vertices was smaller than this threshold, the spontaneous activity map was weighted more heavily than the task-activation map according to Eqn. 1.

Signal to Noise Calculation for fMRI Data

To compare signal to noise ratios between processing approaches, fMRI time courses were extracted from electrophysiologically-defined hand and tongue regions of interest (ROIs) after the fMRI data had been corrected for linear drift and movement. The signal was defined as the amount of fMRI variance utilized in computing a given map while noise was any residual variance not utilized in map creation. Variance utilized in task-activation mapping was computed based on the hemodynamically convolved task model. Variance utilized in spontaneous activity mapping was computed as variance of spontaneous activity multiplied by the square of the correlation coefficient between the regional timecourse and the somatomotor ROI in the opposite hemisphere. Differences in signal to noise ratio between mapping strategies was compared using a Wilcoxon paired non-parametric test.

Comparing the Task fMRI and Combination fMRI Mapping with ECS Findings

Results of different mapping modalities were projected to each individual's brain surface for the comparison with the ECS findings. Taking the ECS findings as the reference, sensitivity and specificity of the activation map and combination map were quantified. Sensitivity was computed by dividing the number of true positives (fMRI positive vertices that were also positive by ECS) by the number of true positives plus false negatives (i.e. total vertices positive by ECS). The specificity was computed by the number of true negatives (fMRI negative vertices that were also negative by ECS) divided by the number of true negatives plus false positives (i.e. total vertices negative by ECS). Receiver operating characteristic (ROC) curves were obtained by calculating the sensitivity and specificity across a wide range of different thresholds. The area under the curve was computed for each subject and compared across methods using a Wilcoxon paired non-parametric test.

### Alternative Approaches

In addition to the combination mapping approach detailed above, several alternative approaches to combination mapping were also tested, and their performance compared to that of the primary analysis. In all cases, task activation maps were combined with other maps that could contain useful information for functional localization, using a selective weighting scheme as detailed above. In particular, rather than combining task-activation maps with spontaneous activity maps, the task-activation map could be combined with anatomical information to improve accuracy. This approach is similar to processes clinicians use when examining conventional pre-operative fMRI maps, where greater emphasis is placed on activations close to the expected anatomical location than those distant from it. Thus, two anatomical weighting approaches were used. In the first approach, the automatic anatomical parcellation generated by Freesurfer surface registration was used as subject-specific masks of the pre and post-central gyri. In the second approach, the putative hand and tongue seed regions obtained by the functional connectivity analysis were employed as anatomical masks. In each case, hybrid or combination maps were produced using the same weighting algorithm detailed above, but combining task-activation maps with anatomical maps rather than the maps based on underlying spontaneous activity.

Furthermore, in six of eight subjects, pure resting state data was collected for comparison with spontaneous activity decomposed from the task runs, as described. As such, a similar selective weighting was performed using spontaneous activity parcellation maps generated based on true resting state data from each subject. This analysis provided information regarding whether there is benefit to acquiring separate resting state and task activation scans to generate the two maps or whether both maps can be generated using the same dataset as proposed here.

For all alternative combination mapping strategies, ROC curves were generated and the area under the curve was compared with that the primary combination mapping analysis.

### Results

In the motor regions of interest defined by ECS, task-related activity accounted for 32.5% of the total variance in the BOLD signal. In traditional task-activation mapping, the rest of the BOLD variance including coherent spontaneous activity is discarded as noise. In the combination mapping approach described herein, this coherent spontaneous activity is not discarded, but used as an additional signal for functional mapping along with the task-related activity. On average, combination mapping results in a 43.2% increase in signal to noise ratio (p<0.001) compared to the conventional task activation approach. However, this improvement was highly variable between subjects, ranging from 26.8% to 74.9% (Table 1).

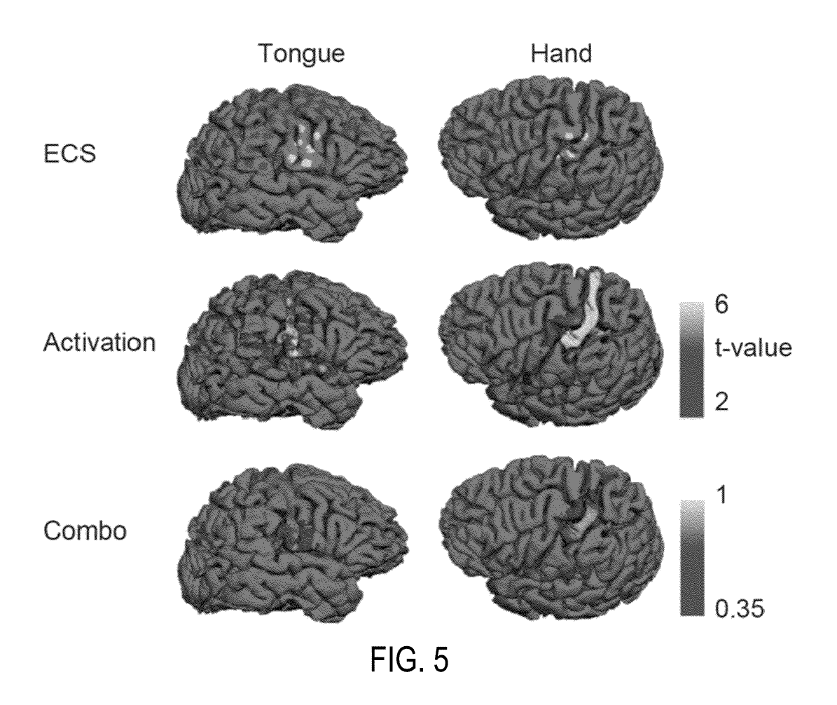

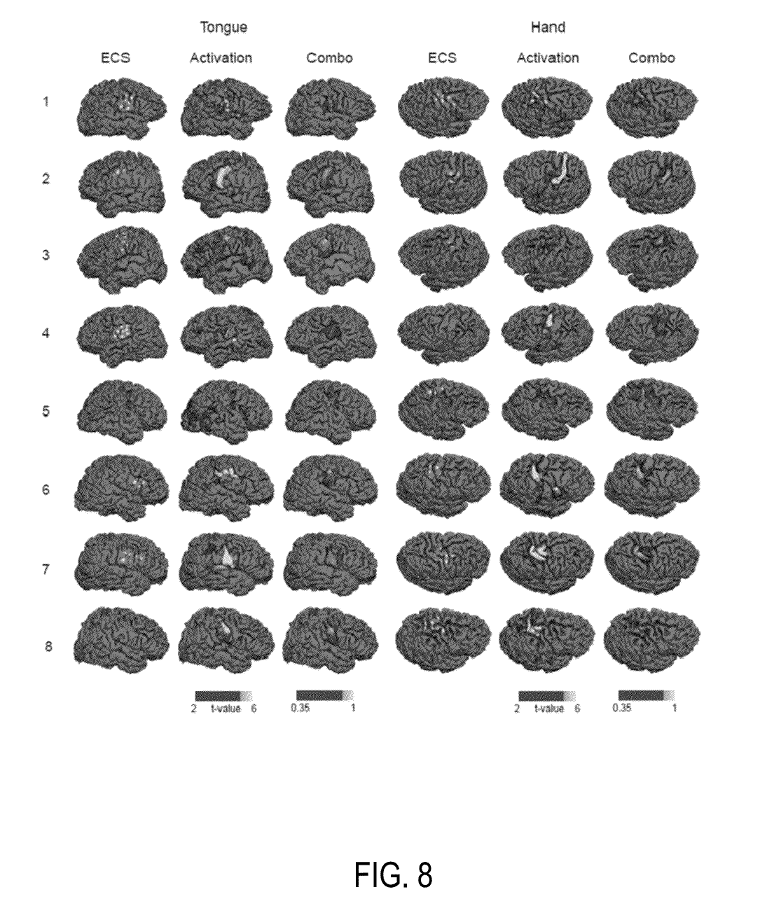

Next, we determined whether this improvement in signal to noise ratio actually translated into more accurate pre-operative maps, using electrical cortical stimulation as the gold standard (FIG. 5, first row). Conventional task-activation maps often implicated regions outside the sensorimotor strip including portions of the temporal and occipital lobes (FIG. 5, second row, and also FIG. 8). Combining the task-activation map with the map based on underlying spontaneous activity appeared to improve specificity and correspondence to results obtained with cortical stimulation (FIG. 5, bottom row).

TABLE 1
Comparison of changes in signal power, noise power, and signal-
to-noise ratio with combination mapping with respect to conventional
task-activation mapping for each subject.

| Subject | % Signal Change | % Noise Change | % SNR Change |
|---|---|---|---|
| 1 | 20.9*** | −15.2*** | 42.6*** |
| 2 | 13.5** | −12.9*** | 30.4*** |
| 3 | 41.3*** | −11.1*** | 59.0*** |
| 4 | 30.0*** | −10.0*** | 44.5*** |
| 5 | 64.0*** | −6.2** | 74.9*** |
| 6 | 23.2*** | −7.6*** | 33.5*** |
| 7 | 10.9*** | −12.5*** | 26.8*** |
| 8 | 20.1*** | −10.1*** | 33.8*** |
| Avg | 28.0*** | −10.7*** | 43.2*** |

**p < .01,
***p < .001,
Wilcoxon paired non-parametric test

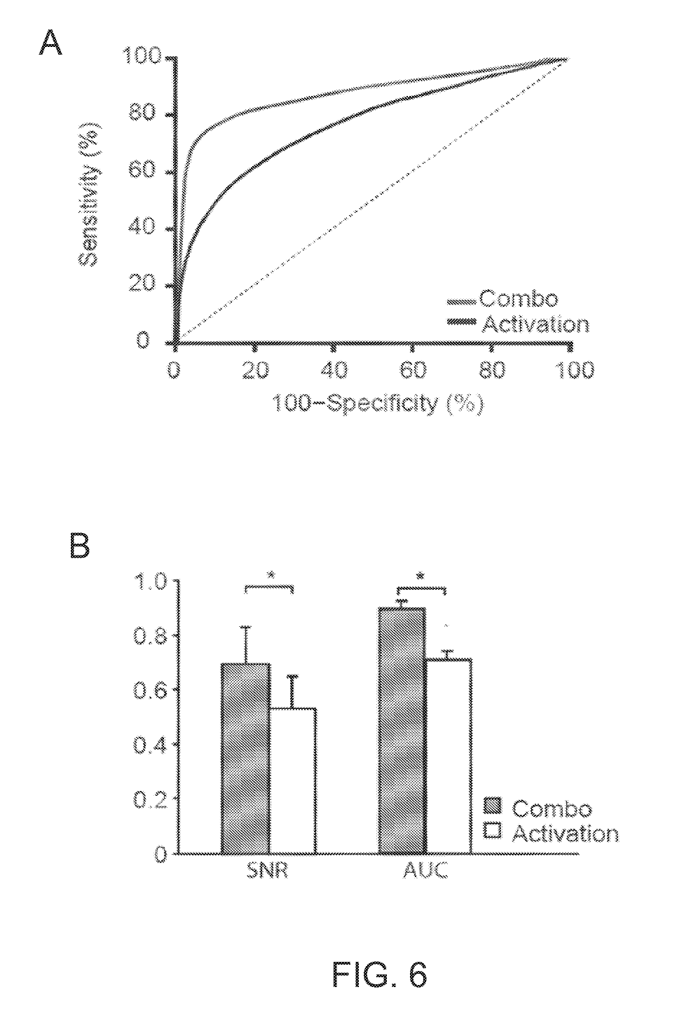

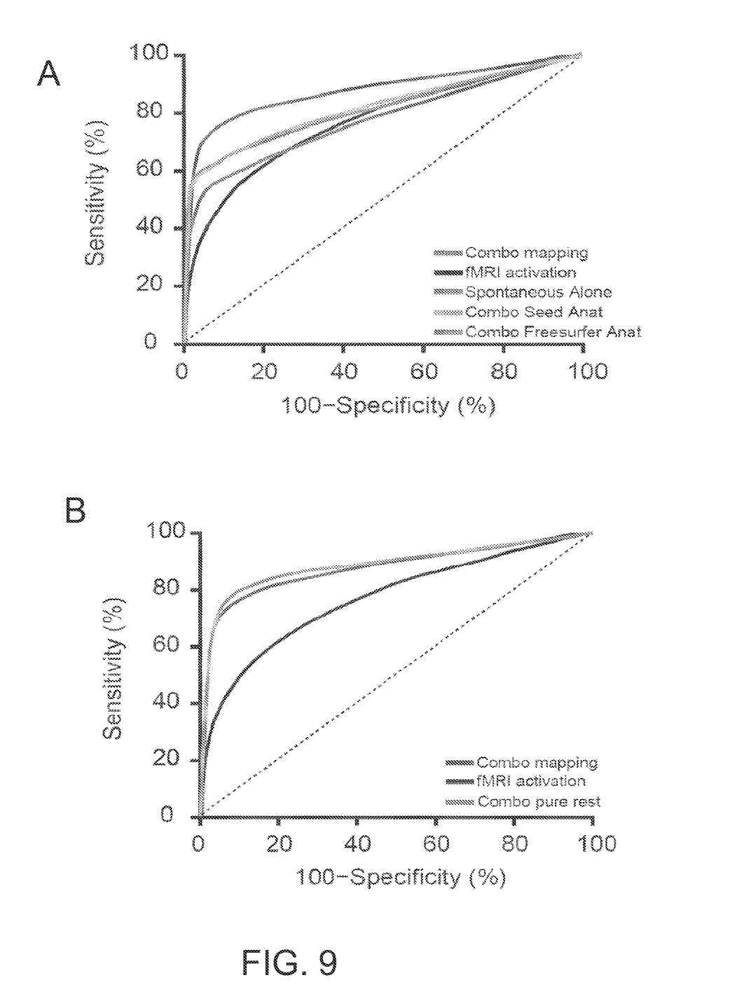

To quantitatively compare the performance of combination mapping with traditional task activation mapping, independent of threshold, we constructed ROC curves then averaged these curves across subjects (FIG. 6A,B). ROC curves indicate the sensitivity and specificity of a technique across different thresholds. For example, if one were to threshold maps for a specificity of 80% combination mapping would improve sensitivity from 62% to 82% compared to task activation mapping. If one were to threshold for a sensitivity of 70%, combination mapping would increase specificity from 71% to 95% compared to task activation mapping. A larger area under the curve (AUC) indicates a more sensitive and specific technique across all thresholds. Combination mapping showed a significant improvement over conventional task activation mapping (AUC of 0.890 vs 0.767, p<0.01) (FIG. 6A,B, see also Table 2). Those subjects that showed the largest improvement in signal to noise with combination mapping also showed the largest improvement in AUC. In fact, the two metrics were highly correlated (r=0.92, p<0.005) providing a nice internal validation of the two quantification approaches. Note that this improvement came from the combination of the two mapping approaches, not just the accuracy of the spontaneous activity map alone, as the combination map performed significantly better than spontaneous activity by itself (AUC of 0.890 vs 0.757, p<0.01) (FIG. 9A).

An important question is whether the spontaneous activity mapping is contributing anything to the combination map beyond simple anatomical weighting. We recomputed combination maps based on anatomical weighting (see methods). The anatomy-weighted combination map showed some improvement in AUC (0.81 for Freesurfer anatomical parcellation and 0.82 for pre-determined seeds) beyond the conventional task activation approach (p<0.01 for both approaches), but was significantly worse than our main combination mapping approach using underlying spontaneous activity (p<0.01 for both approaches) (FIG. 9A, see also Table 2).

TABLE 2
Performance comparison between combination mapping and
traditional task activation mapping.

| | Task- | Spontaneous | | Combo | Combo | Combo |
|---|---|---|---|---|---|---|
| | Activation | Activity | Combo | (Pure | (Freesurfer | (Seed |
| Subject | Alone | Alone | Map | Rest) | Anatomy) | Anatomy) |
| 1 | 74.12 | 70.48 | 83.88 | | 78.03 | 78.02 |
| 2 | 95.37 | 86.37 | 98.12 | 94.62 | 97.41 | 96.62 |
| 3 | 72.64 | 75.23 | 90.93 | 91.12 | 78.61 | 82.10 |
| 4 | 68.49 | 82.01 | 89.09 | 93.93 | 74.53 | 79.04 |
| 5 | 64.63 | 74.62 | 92.68 | | 74.86 | 79.12 |
| 6 | 69.62 | 70.63 | 77.94 | 79.33 | 72.08 | 71.26 |
| 7 | 86.22 | 71.93 | 87.31 | 85.50 | 87.65 | 87.12 |
| 8 | 82.51 | 74.19 | 92.37 | 95.46 | 87.12 | 85.60 |
| Average | 76.70 | 75.68 | 89.04 | 89.99 | 81.29 | 82.36 |

Combo, Task-Activation p < 0.01
Combo, Combo (Pure Rest) p > 0.65
Combo, Combo (Freesurfer Anat) p < 0.02
Combo, Combo (Seed Anat) p < 0.01
Combo, Spont Activity Alone p < 0.01
Combo (Freesurfer Anat), Activation p < 0.01
Combo (Seed Anat), Activation p < 0.01
p values are based on Wilcoxon paired non-parametric test

Another important question is whether combination mapping would be even better if one used a dedicated resting state fMRI scan to compute the spontaneous activity map rather than the spontaneous activity underlying the task-based signal. ROC curves constructed using these two approaches were nearly indistinguishable (AUC 0.890 vs 0.899, p>0.65) (FIG. 9B, see also Table 2), suggesting that there may be lessened benefit to collecting a dedicated resting state scan and potentially doubling scan duration.

### Discussion

Prior work has shown that coherent spontaneous activity does not disappear during task paradigms, but continues. To a rough approximation, there is a linear superposition between task-evoked and spontaneous activity, although some interaction between these two types of activity may occur. Although only the task-evoked signal components are routinely used for functional mapping, the present invention recognizes that one can advantageously combine maps generated using task as well as non-task activity signals.

Thus far, it has been unclear whether functional maps generated using underlying spontaneous activity contain useful information for functional localization beyond the information already available from task-evoked maps. In addition, it has also been unknown whether such information would be advantageous in comparison to information obtained from anatomical data or “pure” spontaneous activity recorded during an independent resting state scan. As such, the present disclosure has provided a first examination of these questions, in part on account of comparison with gold standard data, and analysis techniques capable of assessing the sensitivity and specificity of different fMRI mapping approaches.

In summary, the present invention provides a system and methods that implement a novel approach to processing fMRI data, which greatly improves signal to noise, sensitivity, and specificity, compared to other conventional brain mapping approaches. In particular, the present technique relies on the premise that fMRI signals recorded during a functional task paradigm are composed of task-based modulation and underlying coherent spontaneous, non-task, activity, and both signals, when properly separated, provide advantageous information for functional brain mapping. Additionally, combination mapping methods presented make use of a selective weighting approach driven by subject data to combine task and non-task maps. In particular, a robustness of task activity mapping is utilized to determine a weighting between task and non-task maps. Results shown herein have implications for clinical practice as well as providing insight into the relationship between spontaneous and task-evoked brain activity.

The approach of the present invention includes several aspects that make it broadly applicable. For example, spontaneous, or non-task, activity mapping may only dependent on correlations assessed between remote regions of interest, where in the case somatomotor cortex, correlations are in opposite hemispheres. This makes the presented approach robust to local disturbances in anatomy, such as due to brain tumors. In addition, the present invention allows for adjustment to varying levels of ability in performing a functional task, defaulting to complete spontaneous, or non-task, activity mapping in a patient with no task activation. This is advantageous for pre-operative mapping patients for whom it may be difficult to predict task compliance a priori. Moreover, results using the present invention have demonstrated the greatest benefit in patients with the poorest activation. This is promising for language or memory mapping which often produces a less robust activation map compared to motor mapping.

Another advantage of the systems and methods described here our algorithm is that they do not require additional tasks or scan time to improve functional localization. Other approaches such as adding additional tasks or performing separate task and rest scans may be limited by these practical considerations.

Still another advantage of the systems and methods described here our algorithm is that they are designed to adjust to varying levels of ability to perform the task, defaulting to complete spontaneous activity mapping in a patient with no task activation. This is an advantage for impaired patient populations in whom predicting task compliance a priori may be difficult. Finally, the spontaneous activity mapping is only dependent on correlations assessed with remote regions of interest, which should make the systems and methods of the present invention robust to local disturbances in anatomy like brain tumors.

Features suitable for such combinations and sub-combinations would be readily apparent to persons skilled in the art upon review of the present application as a whole. The subject matter described herein and in the recited claims intends to cover and embrace all suitable changes in technology.

1. A method for functional brain mapping, the method comprising:
i. providing a set of time-series functional magnetic resonance imaging (fMRI) data acquired with a magnetic resonance imaging (MRI) system from a brain of a subject while the subject performed a functional task;
ii. decomposing the set of time-series fMRI data into a set of task signals indicative of neuronal activity associated with the functional task performed by the subject and a set of non-task signals indicative of spontaneous neuronal activity using a model related to the functional task performed by the subject;
iii. generating a task activity map using the set of task signals;
iv. generating a non-task activity map using the set of non-task signals; and
v. producing a combination map by:
selectively weighting the task activity map using a weighting value based on a statistical parameter derived from the task activity map;
selectively weighting the non-task activity map using a different weighting value based on the statistical parameter derived from the task activity map; and
combining the selectively weighted task activity map with the selectively weighted non-task activity map.

2. The method of claim 1, wherein the model is a general linear model.

3. The method of claim 1, wherein the statistical parameter is a median t-value computed, using the task activity map, for a plurality of locations in a region of interest in the brain of the subject.

4. The method of claim 1, wherein the statistical parameter is a mean t-value computed, using the task activity map, for a plurality of locations in a region of interest in the brain of the subject.

5. The method of claim 1, wherein the task activity map and non-task activity map are equally weighed.

6. The method of claim 1, wherein the task activity map and non-task activity map are weighed according to:
$$CM = ( 1 - \frac{T}{2 \cdot th} ) \times SAM + ( \frac{T}{2 \cdot th} ) \times TAM ;$$
wherein CM is the combination map; SAM is the non-task activity map; TAM is the task-activity map; T is the statistical parameter; and th is a threshold value.

7. The method of claim 1, wherein the method further comprises generating, using the combined map, a report indicative at least a region of interest in the brain of the subject.

8. A system for functional brain mapping the system comprising:
an input configured to receive a set of time-series functional magnetic resonance imaging (fMRI) data acquired from a brain of a subject while performing an activity;
at least one processor configured to:
a. decompose the set of time-series fMRI data into a set of task signals indicative of neuronal activity associated with the functional task performed by the subject and a set of non-task signals indicative of spontaneous neuronal activity using a model related to the functional task performed by the subject;
b. generate a task activity map using the set of task signals;
c. generate a non-task map using the set of non-task signals;
d. produce a combination map by:
selectively weighting the task activity map using a weighting value based on a statistical parameter derived from the task activity map;
selectively weighting the non-task activity map using a different weighting value based on the statistical parameter derived from the task activity map;
combining the selectively weighted task activity map with the selectively weighted non-task activity map;
e. generate, using the combined map, a report indicative a region of interest in the brain of the subject; and
an output configured to display the report.

9. The system of claim 8, wherein the model is a general linear model.

10. The system of claim 8, wherein the statistical parameter is a median t-value computed, using the task activity map, for a plurality of locations in a region of interest in the brain of the subject.

11. The system of claim 8, wherein the statistical parameter is a mean t-value computed, using the task activity map, for a plurality of locations in a region of interest in the brain of the subject

12. The system of claim 8, wherein the at least one processor is further configured to produce the combination map by equally weighting the task activity map and non-task activity map.

13. The system of claim 8, wherein the at least one processor is further configured to produce the combination map by weighting the task activity map and non-task activity map according to:
$$CM = ( 1 - \frac{T}{2 \cdot th} ) \times SAM + ( \frac{T}{2 \cdot th} ) \times TAM ;$$
wherein CM is the combination map; SAM is the non-task activity map; TAM is the task-activity map; T is the statistical parameter; and th is a threshold value.

14. A method for functional brain mapping, the method comprising:
i. providing a set of time-series functional magnetic resonance imaging (fMRI) data acquired using a magnetic resonance imaging (MRI) system from a brain of a subject while the subject performed a functional task;
ii. processing the set of time-series fMRI data using a model related to the functional task performed by the subject to separate a set of task signals indicative of neuronal activity associated with the functional task performed by the subject from other signals in the time-series fMRI data;
iii. generating a task activity map using the set of task signals;
iv. providing an image of the subject; and
v. producing a combination map by:
selectively weighting the task activity map using a weighting value based on a statistical parameter derived from the task activity map;
selectively weighting the provided image using a different weighting value based on the statistical parameter derived from the task activity map; and
combining the selectively weighted task activity map with the selectively weighted image.

15. The method of claim 14, wherein the model is a general linear model.

16. The method of claim 14, wherein the provided image of the subject is an anatomical map that depicts anatomy.

17. The method of claim 14, wherein the provided image is a non-task activity map that depicts spontaneous neuronal activity in the subject.

18. The method of claim 14, wherein the statistical parameter is a median t-value computed, using the task activity map, for a plurality of locations in a region of interest in the brain of the subject.

19. The method of claim 14, wherein the statistical parameter is a mean t-value computed, using the task activity map, for a plurality of locations in a region of interest in the brain of the subject.

20. The method of claim 14, wherein the task activity map and the provided image of the subject are weighted according to:
$$CM = ( 1 - \frac{T}{2 \cdot th} ) \times SAM + ( \frac{T}{2 \cdot th} ) \times TAM ;$$
wherein CM is the combination map; I is the provided image of the subject; TAM is the task-activity map; T is the statistical parameter; and th is a threshold value.
